(19) United States
(12) Patent Application Publication
(10) Pub. No.: US 2018/0321257 A1
(43) Pub. Date: Nov. 8, 2018

(54) USE OF CD146 AS A MARKER OF THE VASCULAR WALL TENSION

(71) Applicant: MYCARTIS N.V., Gent (BE)

(72) Inventors: Alexandre Mebazaa, Montrouge (FR); Mattia Arrigo, Paris (FR)

(21) Appl. No.: 15/770,746

(22) PCT Filed: Nov. 7, 2016
(86) PCT No.: PCT/EP2016/076862

§ 371 Date: Apr. 24, 2018

(30) Foreign Application Priority Data
Nov. 6, 2015 (EP) 15193565.7

(51) Int. Cl. G01N 33/68; G01N 33/569; G01N 33/543

(52) U.S. Cl. CPC G01N 33/6893; G01N 33/56966; G01N 33/54366; G01N 2800/329; G01N 2800/326; G01N 2800/325; G01N 2800/321; G01N 2800/324; G01N 2333/70596

(57) ABSTRACT

The present invention concerns the use of CD146 as a marker of the vascular wall tension, especially for diagnosing, predicting and/or prognosticating diseases associated with variations of the vessel wall tension.

### FIELD OF THE INVENTION

The invention relates to protein- and/or peptide-based markers (also named biomarkers) and to agents specifically binding thereto, for use in predicting, diagnosing, prognosticating and/or monitoring diseases or conditions in subjects. More particularly, the application discloses CD146 as a new biomarker for diseases associated with variations of the vascular wall tension; methods for predicting, diagnosing and/or prognosticating said diseases based on measuring said marker; and kits and devices for measuring said marker and/or performing said methods.

### BACKGROUND OF THE INVENTION

In many diseases and conditions, a favourable outcome of prophylactic and/or therapeutic treatments is strongly correlated with early and/or accurate prediction, diagnosis and/or prognosis of the disease or condition. Therefore, there exists a continuous need for additional and preferably improved means and methods for early and/or accurate prediction, diagnosis and/or prognosis of diseases and conditions to guide the treatment choices.

Heart failure (HF) is one of the leading causes of mortality in Western countries and is associated with substantial morbidity (Go A S et al. Heart disease and stroke statistics—2014 update: a report from the American Heart Association. Circulation. 2014; 129:e28-e292). Hospital readmissions, mostly due to the presence of organ congestion, are particularly frequent in patients with chronic heart failure (CHF) and are associated with adverse outcome (Gheorghiade M et al., Am J Cardiol. 2005; 96:11G-17G).

The congestive cascade, which often begins several days or weeks before symptom onset, includes a sub-clinical increase of ventricular filling pressures (“hemodynamic congestion”) which may further lead to redistribution of fluids within the lungs and visceral organs (“organ congestion”) and finally to overt signs and symptoms of volume overload (“clinical congestion”) (Picano E et al., Heart Fail Rev. 2010; 15:63-72). Several strategies for early detection of sub-clinical organ congestion have been proposed, e.g. daily body weight measurement, or more recently, intrathoracic impedance monitoring and implantable hemodynamic monitoring (Bourge R C et al., J Am Coll Cardiol. 2008; 51:1073-1079; Hindricks G et al., Lancet. 2014; 384:583-590; Abraham W T et al., Lancet. 2011; 377:658-666).

However, there is still an unmet need for reliable and non-invasive detection of congestion at an early hemodynamic stage in order to, hopefully, reduce hospitalizations and improve outcome.

The use of biomarkers, in particular natriuretic peptides (NPs), was advocated for this purpose (McMurray J J V et al., European Heart Journal. 2012; 33:1787-1847). However, levels of circulating NPs are influenced by a broad range of cardiac abnormalities including myocardial stretch and cell necrosis and may therefore not accurately reflect peripheral congestion (Sabatine M S et al., J Am Coll Cardiol. 2004; 44:1988-1995).

In view of this, there exists a persistent need for additional and preferably specific biomarkers for monitoring congestion, i.e. overfilling and distention of the vessels with blood.

### BRIEF SUMMARY OF THE INVENTION

Having conducted extensive experiments and tests, the inventors have revealed that melanoma cell adhesion molecule (MCAM, also known as CD146 or MUC18) represents a new biomarker of the peripheral congestion. More generally, it is a marker of the vascular (vessel) wall tension (stretch) and is particularly advantageous for predicting, diagnosing, monitoring and/or prognosticating diseases associated with variations of the vascular wall tension.

### Definitions

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The terms “comprising”, “comprises” and “comprised of” as used herein are synonymous with “including”, “includes” or “containing”, “contains”, and are inclusive or open-ended and do not exclude additional, non-recited members, elements or method steps.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Homologous” or “identical” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous or identical at that position. The percent of homology/identity between two sequences is a function of the number of matching or homologous positions shared by the two sequences divided by the number of positions compared ×100. For example, if 6 of 10 of the positions in two sequences are matched or homologous then the two sequences are 60% homologous/identical. Generally, a comparison is made when two sequences are aligned to give maximum homology/identity.

The term “isolated” with reference to a particular component (such as for instance, a protein, polypeptide, peptide or fragment thereof) generally denotes that such component exists in separation from—for example, has been separated from or prepared in separation from—one or more other components of its natural environment. For instance, an isolated human or animal protein, polypeptide, peptide or fragment exists in separation from a human or animal body where it occurs naturally.

The term “isolated” as used herein may preferably also encompass the qualifier “purified”. As used herein, the term “purified” with reference to protein(s), polypeptide(s), peptide(s) and/or fragment(s) thereof does not require absolute purity. Instead, it denotes that such protein(s), polypeptide(s), peptide(s) and/or fragment(s) is (are) in a discrete environment in which their abundance (conveniently expressed in terms of mass or weight or concentration) relative to other proteins is greater than in a biological sample. A discrete environment denotes a single medium, such as for example a single solution, gel, precipitate, lyophilisate, etc. Purified peptides, polypeptides or fragments may be obtained by known methods including, for example, laboratory or recombinant synthesis, chromatography, preparative electrophoresis, centrifugation, precipitation, affinity purification, etc. Purified protein(s), polypeptide(s), peptide(s) and/or fragment(s) may preferably constitute by weight 10%, more preferably 50%, such as 60%, yet more preferably 70%, such as 80%, and still more preferably 90%, such as 95%, 96%, 97%, 98%, 99% or even 100%, of the protein content of the discrete environment. Protein content may be determined, e.g., by the Lowry method (Lowry et al. 1951. J Biol Chem 193: 265), optionally as described by Hartree 1972 (Anal Biochem 48: 422-427). Also, purity of peptides or polypeptides may be determined by SDS-PAGE under reducing or non-reducing conditions using Coomassie blue or, preferably, silver stain.

The term “biomarker” is widespread in the art and may broadly denote a biological molecule and/or a detectable portion thereof whose qualitative and/or quantitative evaluation in a subject is predictive or informative (e.g., predictive, diagnostic and/or prognostic) with respect to one or more aspects of the subject's phenotype and/or genotype, such as, for example, with respect to the status of the subject as to a given disease or condition.

The terms “predicting” or “prediction”, “diagnosing” or “diagnosis” and “prognosticating” or “prognosis” are commonplace and well-understood in medical and clinical practice.

By means of further explanation and without limitation, “predicting” and “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction of a disease” in a subject may also particularly mean that the subject has a “positive” prediction of said disease, i.e., that the subject is at risk of having said disease (e.g., the risk is significantly increased vis-à-vis a control subject or subject population).

The terms “diagnosing” or “diagnosis” generally refer to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). As used herein, “diagnosis of a disease” in a subject may particularly mean that the subject has said disease, hence, is diagnosed as having said disease. A subject may be diagnosed as taught herein as not having said disease despite displaying one or more conventional symptoms or signs reminiscent thereof.

The terms “prognosticating” or “prognosis” generally refer to an anticipation on the progression of a disease or condition and the prospect (e.g., the probability, duration, and/or extent) of recovery. A good prognosis of a disease may generally encompass anticipation of a satisfactory partial or complete recovery from said disease, preferably within an acceptable time period. A good prognosis of said disease may more commonly encompass anticipation of not further worsening or aggravating of the conditions, preferably within a given time period. A poor prognosis of a disease may generally encompass anticipation of a substandard recovery and/or unsatisfactorily slow recovery, or to substantially no recovery or even further worsening of said disease.

A molecule or analyte such as a protein, polypeptide or peptide, or a group of two or more molecules or analytes such as two or more proteins, polypeptides or peptides, is “measured” in a sample when the presence or absence and/or quantity of said molecule(s) or analyte(s) is detected or determined in the sample, preferably substantially to the exclusion of other molecules and analytes.

The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

A relative quantity of a molecule or analyte in a sample may be advantageously expressed as an increase or decrease or as a fold-increase or fold-decrease relative to said another value, such as relative to a reference value as taught herein. Performing a relative comparison between first and second parameters (e.g., first and second quantities) may but need not require to first determine the absolute values of said first and second parameters. For example, a measurement method can produce quantifiable readouts (such as, e.g., signal intensities) for said first and second parameters, wherein said readouts are a function of the value of said parameters, and wherein said readouts can be directly compared to produce a relative value for the first parameter vs. the second parameter, without the actual need to first convert the readouts to absolute values of the respective parameters.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The terms “patient,” “subject,” “individual,” and the like are used interchangeably herein, and refer to any animal, or cells thereof whether in vitro or in situ, amenable to the methods described herein. As used herein they typically denote humans, but may also encompass reference to non-human animals, preferably warm-blooded animals, more preferably mammals, such as, e.g., non-human primates, rodents, canines, felines, equines, ovines, porcines, and the like.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced. A disease or disorder is “cured” if the severity of a symptom of the disease or disorder, the frequency with which such a symptom is experienced by a patient, or both, is eliminated.

A “therapeutic” treatment is a treatment administered to a subject who exhibits signs of pathology, for the purpose of diminishing or eliminating those signs.

As used herein, “treating a disease or disorder” means reducing the frequency or severity of at least one sign or symptom of a disease or disorder experienced by a subject. Disease and disorder are used interchangeably herein in the context of treatment.

An “effective amount” of a compound is that amount of compound which is sufficient to provide a beneficial effect to the subject to which the compound is administered. The phrase “therapeutically effective amount,” as used herein, refers to an amount that is sufficient or effective to prevent or treat (delay or prevent the onset of, prevent the progression of, inhibit, decrease or reverse) a disease or disorder or condition, including alleviating symptoms thereof. An “effective amount” of a delivery vehicle is that amount sufficient to effectively bind or deliver a compound.

### DETAILED DESCRIPTION OF THE INVENTION

According to a first aspect, the present invention concerns the use of CD146 as a marker of the vascular wall tension.

As used herein, the term “CD146” corresponds to the protein commonly known as Melanoma Cell Adhesion Molecule (MCAM) or MUC18, i.e. the proteins and polypeptides commonly known under these designations in the art. The terms encompass such proteins and polypeptides of any organism where found, and particularly of animals, preferably vertebrates, more preferably mammals, including humans and non-human mammals, even more preferably of humans. The terms particularly encompass such proteins and polypeptides with a native sequence, i.e., ones of which the primary sequence is the same as that of CD146 found in or derived from nature. A skilled person understands that native sequences of CD146 may differ between different species due to genetic divergence between such species. Moreover, the native sequences of CD146 may differ between or within different individuals of the same species due to normal genetic diversity (variation) within a given species. Also, the native sequences of CD146 may differ between or even within different individuals of the same species due to post-transcriptional or post-translational modifications. Accordingly, all CD146 sequences found in or derived from nature are considered “native”. The terms encompass CD146 proteins and polypeptides when forming a part of a living organism, organ, tissue or cell, when forming a part of a biological sample, as well as when at least partly isolated from such sources. The terms also encompass proteins and polypeptides when produced by recombinant or synthetic means.

Exemplary CD146 includes, without limitation, human CD146 having primary amino acid sequence as annotated under Uniprot/Swissprot (http://www.expasy.org/) accession number NP_006491, as shown in FIG. 1 (SEQ ID NO: 1) of WO 2011/048168. A skilled person can also appreciate that said sequences are of precursor of CD146 and may include parts which are processed away from mature CD146. For example, the CD146 protein can be in a soluble form or can be attached to the cell membrane. In FIG. 1 of WO 2011/048168, the signal peptide and transmembrane and cytoplasmic domains are indicated in small caps in the amino acid sequence. Also indicated is the selected MASSterclass quantified peptide (pept25—bold, underlined: SEQ ID NO: 2 of WO 2011/048168). This MASSterclass peptide can quantify both the full length and cleaved soluble form of CD146, although due to the experimental set-up only the plasma circulating fraction (i.e. the non-cell bound fraction) is measured.

The CD146 protein is specific for endothelial cells and vascular smooth muscle cells and has been used as a tool for sorting endothelial cells out of a population of blood cells, based on the membrane bound form of CD146. CD146 belongs to the immunoglobulin supergene family with five immunoglobulin like domains (V-V-C2-C2-C2), a transmembrane region and a 63 residue cytoplasmic tail. It is a membrane glycoprotein that functions as a Ca²⁺ independent cell adhesion molecule involved in heterophilic cell to cell interactions. The protein has a molecular size of 130 kDa in its reduced form (118 kDa unreduced), and N linked glycosylation accounts for fifty percent of the apparent molecular weight. Soluble CD146 is released by ectodomain shedding (through the action of MMPs). Increased plasma levels of soluble CD146 was observed in patients with chronic renal failure (Healthy serum levels: 270 ng/ml; renal failure patients: 500 ng/ml) as discussed in Saito et al., 2008 (Clin Exp Nephrol. 2008 February; 12(1):58-64. Epub 2008 Jan. 5). On the other hand, decreased serum levels of sCD146 (soluble CD146) were observed in patients with Inflammatory Bowel Disease (IBD) such as Crohn's disease, while the membrane bound CD146 expression is increased in active IBD (Bardin et al., Inflamm. Bowel Dis. 2006 January; 12(1):16-21 and Reumaux et al., Inflamm. Bowel Dis. 2007 October; 13(10):1315-7).

In a preferred embodiment, the circulating CD146 protein, e.g. the form circulating in the blood plasma, is used, as opposed to the membrane- or cell-bound CD146 protein (e.g. CD146 present on the endothelial cell surface).

Whereas CD146 has been known as an endothelial cell injury marker, said marker is often used as a tool for sorting endothelial cells, implying the membrane bound (full-length) protein is used (cf. e.g. WO 2006/020936). WO 2011/048168 has shown that this marker is useful to distinguish AHF patients from patients having dyspnea due to other causes (non-AHF patients).

The reference herein to CD146 may also encompass fragments of CD146. Hence, the reference herein to measuring CD146, or to measuring the quantity of CD146, may encompass measuring the CD146 protein or polypeptide, such as, e.g., measuring the mature and/or the MMP-processed soluble form (shortly called “soluble form” hereinafter) of CD146 and/or measuring one or more fragments thereof. For example, CD146 and/or one or more fragments thereof may be measured collectively, such that the measured quantity corresponds to the sum amounts of the collectively measured species. In another example, CD146 and/or one or more fragments thereof may be measured each individually. Preferably, said fragment of CD146 is a plasma circulating form of CD146. The expression “plasma circulating form of CD146” or shortly “circulating form” encompasses all CD146 proteins or fragments thereof that circulate in the plasma, i.e. are not cell- or membrane-bound. Without wanting to be bound by any theory, such circulating forms can be derived from the full-length CD146 protein through natural processing (e.g. MMP-cleavage into its “soluble form” as indicated above), or can be resulting from known degradation processes occurring in said sample. In certain situations, the circulating form can also be the full-length CD146 protein, which is found to be circulating in the plasma. Said “circulating form” can thus be any CD146 protein or any processed soluble form of CD146 or fragments of either one, that is circulating in the sample, i.e. which is not bound to a cell- or membrane fraction of said sample.

As used therein, the term “vascular” refers to the vessels, and include arteries, veins and capillaries forming the vascular system in subjects, preferably in humans. According to a specific embodiment, the present invention concerns the peripheral vascular system.

The terms “wall tension” or “wall stretch” are considered to have a similar meaning in the frame of the invention. They can be evaluated physically by methods known in the art, e.g. by evaluating the pressure or the diameter of the vessels, e.g. using catheters or imaging (especially echography) analysis.

In the frame of the invention, it has been demonstrated that CD146 is a marker of the vascular wall tension. Therefore, it can be used to evaluate the vascular wall tension or to monitor variations thereof. Whereas the measurement of said marker is easy, especially via in vitro methods performed on a sample of a subject, rapid, reliable and cost-effective, it can replace the direct measurement of wall tension via in vivo invasive methods.

In the frame of the invention, it has been shown that there is a direct correlation between the level of CD146 and the status of the vascular wall tension: In case of vascular stress, i.e. when the wall tension or stretch (and possibly the pressure and/or the diameter of the vessel) is increased, there is an increased release of CD146, especially of soluble CD146, and then an increased level of CD146.

According to one aspect, the present invention concerns CD146 for use in a method for diagnosing, predicting and/or prognosticating diseases associated with variations of the vascular wall tension in a subject. In others words, the invention provides a (in vivo) method for diagnosing, predicting and/or prognosticating a disease associated with variations of the vascular wall tension in a subject, wherein the method comprises measuring the quantity of CD146 in the subject.

According to another aspect, the present invention concerns the use of CD146 as a marker for diagnosing, predicting and/or prognosticating diseases associated with variations of the vascular wall tension in a subject. In others words, the invention provides a (in vitro) method for diagnosing, predicting and/or prognosticating a disease associated with variations of the vascular wall tension in a subject, wherein the method comprises measuring the quantity of CD146 in a sample from the subject.

One understands that methods of prediction, diagnosis, and/or prognosis of diseases or conditions generally comprise an examination phase in which data is collected from and/or about the subject. According to an embodiment, the method of the invention comprises measuring the quantity of CD146 in a sample from the subject.

As demonstrated in the present invention, CD146 is useful for diagnosing, predicting and/or prognosticating diseases or conditions associated with variations of the vascular wall tension.

As explained above, congestion occurs through cascade, which often begins several days or weeks before symptom onset, includes a sub-clinical increase of ventricular filling pressures (“hemodynamic congestion”) which may further lead to redistribution of fluids within the lungs and visceral organs (“organ congestion”) and finally to overt signs and symptoms of volume overload (“clinical congestion”). According to the invention, CD146 is a marker which can be used in relation to any condition associated with a change in the vascular wall tension, especially an increase in the vascular wall tension. The present application clearly establishes a correlation between plasma levels of sCD146 and hemodynamic congestion (right-atrial pressure, left-atrial or pulmonary-artery occlusion pressure). Therefore, it can be considered as a direct marker of hemodynamic congestion but also as an indirect, early marker of further conditions resulting thereof, especially organ congestion.

According to a preferred embodiment, said disease or condition is selected in the following group:
hemodynamic disorders;
organ congestion, advantageously pulmonary congestion or visceral (advantageously kidney or liver) congestion;
mitral valve diseases, advantageously mitral stenosis or mitral regurgitation;
pulmonary hypertension, affecting arteries, veins or capillaries, advantageously pre-capillary pulmonary hypertension.

According to a specific embodiment, the present invention does not concern the use of CD146 as a marker of edema.

According to one aspect, the present invention concerns mitral stenosis (MS). Mitral stenosis typically occurs secondary to rheumatic fever, and consists in a narrowing of the mitral valve opening. If left untreated, mitral stenosis will progress slowly, i.e. the mitral valve opening will grow narrower over time, which may cause complications such as progressive dyspnea on exertion, pulmonary hypertension (PH), atrial fibrillation and right ventricular (RV) failure. Indeed, the present application reports the possibility to use the biomarker CD146 as an alternative for the initial assessment of MS and its complications, instead of a complete cardiologic assessment including ECG and echocardiography. In other terms, among other aspects, the invention enables the early predication of mitral stenosis, at a stage where the subject does not yet presents with clinical signs of the complications.

According to another aspect, the present invention concerns pulmonary congestion. Indeed, the present application reports the possibility to use the biomarker CD146 as an alternative for the assessment of pulmonary congestion, instead of conventional chest radiography, or even more accurate methods for the assessment of left-sided congestion such as non-invasive (flow of the pulmonary vein, lung ultrasound, CT scan) or invasive (pulmonary catheter) methods.

Variations of the vascular wall tension, in particular increase of the vascular wall tension, may be related to variety of diseases or conditions, some of which may later evolve into further clinical complications. The method of the invention may therefore be useful for a variety of subjects, independent of their actual clinical state.

According to one embodiment, the method of the invention is performed on a subject who does not have clinical signs of heart disease, preferably does not have clinical signs of heart failure (HF), advantageously of chronic heart failure (CHF) or of acute coronary syndrome (ACS).

In a preferred embodiment, the method of the invention is for diagnosing, predicting and/or prognosticating a disease associated with variations of the vascular wall tension in a subject, wherein the method comprises measuring the quantity of CD146 in a sample from the subject, wherein said disease is organ congestion, advantageously pulmonary congestion or visceral congestion, and wherein the subject does not have clinical sign of heart disease, preferably wherein the subject has a healthy heart, advantageously wherein the subject has a healthy cardio-pulmonary system.

By “healthy heart”, it is herein referred to a heart devoid of any known pathology. Preferably, a healthy hart is devoid of cardiac disease. By “healthy cardio-pulmonary system”, it is herein referred to a cardio-pulmonary system devoid of any known pathology.

By “visceral congestion”, it is herein referred to the congestion of visceral organs; i.e. of the organs located within the three major bodily cavities: the thorax, the abdomen and the pelvis. The visceral organs of the thorax include the heart and the lungs. The visceral organs of the abdomen include the stomach, the large and small intestines, the pancreas, the kidneys, the appendix, the adrenal gland, the gall bladder, the liver, the spleen and the peritoneum. The visceral organs of the pelvis include the bladder, the ovaries and testicles, the uterus, and the rectum.

Preferably, the term visceral congestion refers to congestion of the visceral organs of the abdomen, and/or of the pelvis.

According to one embodiment, the method of the invention is performed on a subject suffering from heart failure, advantageously from chronic heart failure.

The terms “heart failure” (HF), “acute heart failure” (AHF) and “chronic heart failure” (CHF) as used herein carry their respective art-established meanings. By means of further guidance, the term “heart failure” as used herein broadly refers to pathological conditions characterized by an impaired diastolic or systolic blood flow rate and thus insufficient blood flow from the ventricle to peripheral organs. “Acute heart failure” or also termed “acute decompensated heart failure” may be defined as the rapid onset of symptoms and signs secondary to abnormal cardiac function, resulting in the need for urgent therapy. AHF can present itself acute de novo (new onset of acute heart failure in a patient without previously known cardiac dysfunction) or as acute decompensation of CHF. The term “chronic heart failure” generally refers to a case of heart failure that progresses so slowly that various compensatory mechanisms work to bring the disease into equilibrium. Common clinical symptoms of CHF include inter alia any one or more of breathlessness, diminishing exercise capacity, fatigue, lethargy and peripheral edema. Other less common symptoms include any one or more of palpitations, memory or sleep disturbance and confusion, and usually co-occur with one or more of the above recited common symptoms. In studies such as the present one, CHF population may differ from the AHF population in that CHF patients do not have an acute decompensation and hence do not represent themselves to the ED at the time the clinical sample used in such a study or research is taken. Chronic heart failure patients may, however, easily decompensate leading to “acute heart failure”.

The present invention might bring several benefits in the management of HF patients. Early assessment of the degree of congestion in order to adjust diuretic therapy is not always easy in HF patients. Various tools for early, reliable and non-invasive detection of sub-clinical organ congestion have been proposed, but, despite many efforts, data on body weight measurement, congestion scores, bioimpedance, remain inconclusive. The present application reports that sCD146 is a biomarker of peripheral congestion, and then the diagnostic and prognostic value of sCD146 and its potential to guide decongestive therapy in left and right ventricular HF.

According to one embodiment, the method of the invention is performed on a subject suffering from acute coronary syndrome. Indeed, the present application reports the performance of sCD146 for the assessment of pulmonary congestion in the early phase of ACS.

Acute coronary syndrome (ACS) is a common precipitating factor of acute heart failure (AHF) and the presence of pulmonary congestion in patients with acute coronary syndrome negatively affect short term outcome. Reliable and non-invasive assessment of pulmonary congestion is of importance to select patients in need of more intensive monitoring and therapy. As shown in the present application, CD146 appears as a valuable alternative marker to natriuretic peptides since the levels of circulating natriuretic peptides are influenced by the amount of myocardial ischemia and may not accurately reflect pulmonary congestion in the context of ACS.

According to one embodiment, the method of the invention comprises the following steps:
measuring the quantity of CD146 in the sample from the subject;
comparing the quantity of CD146 measured in the first step with a reference value of a subject not suffering from said disease and
diagnosing, predicting and/or prognosticating said disease in the subject if the quantity is increased or decreased in comparison to the reference value.

In relation to the preferred diseases and conditions listed below, the quantity of CD146 in the sample of the subject suffering from said disease or condition is increased in comparison to the reference value.

According to another embodiment, the method of the invention comprises the following steps:
measuring the quantity of CD146 in the sample from the subject;
comparing the quantity of CD146 measured in the first step with a reference value representing a known diagnosis, prediction and/or prognosis of said disease;
finding a deviation or no deviation of the quantity measured in the first step from the reference value; and
attributing said finding of deviation or no deviation to a particular diagnosis, prediction and/or prognosis of said disease in the subject.

As indicated above, the present methods may employ reference values for the quantity of CD146, which may be established according to known procedures previously employed for other biomarkers. Such reference values may be established either within (i.e., constituting a step of) or external to (i.e., not constituting a step of) the methods of the present invention as defined herein. Accordingly, any one of the methods taught herein may comprise a step of establishing a reference value for the quantity of CD146, said reference value representing either (a) a prediction or diagnosis of no disease or a good prognosis for said disease, or (b) a prediction or diagnosis of the disease or a poor prognosis for said disease.

Hence, in various embodiments, the present methods for predicting, diagnosing and/or prognosticating said diseases or conditions may be used in individuals who have not yet been diagnosed as having said diseases (for example, preventative screening), or who have been diagnosed as having said diseases, or who are suspected of having said diseases (for example, display one or more symptoms characteristic of said diseases), or who are at risk of developing said diseases (for example, genetic predisposition; presence of one or more developmental, environmental or behavioral risk factors).

The methods may also be used to detect various stages of progression or severity of said diseases.

The methods may also be used to detect response to prophylactic or therapeutic treatments or other interventions. In the frame of the application, medical treatments include drug treatment (diuretics drugs, anticoagulants, beta blockers, ACE inhibitors, calcium channel blockers, . . . ) and/or surgical treatment (valve repair, . . . ).

The methods can furthermore be used to help the medical practitioner in deciding upon worsening, status-quo, partial recovery, or complete recovery of the patient from a disease, resulting in either further treatment or observation or in discharge of the patient. The methods of the present invention enable the medical practitioner to monitor the disease progress by measuring the level of CD146 in a sample of the patient, wherein a decrease in CD146 level as compared to a prior CD146 level (e.g. at the time of the admission) indicates the condition of the subject is improving or has improved, while an increase of the CD146 level as compared to the level of CD146 (e.g. as measured upon admission) indicates the condition of the subject has worsened or is worsening.

The invention further provides a method for monitoring a change in the prediction, diagnosis and/or prognosis of said disease in a subject, comprising:
applying the prediction, diagnosis and/or prognosis method as taught here above to the subject at one or more successive time points, whereby the prediction, diagnosis and/or prognosis of said disease in the subject is determined at said successive time points;
comparing the prediction, diagnosis and/or prognosis of said disease in the subject at said successive time points as determined in the first step; and
finding the presence or absence of a change between the prediction, diagnosis and/or prognosis of said disease in the subject at said successive time points as determined in the first step.

This aspect allows to monitor the subject's condition over time. This can inter alia allow to monitor in said subject the disease progression, disease aggravation or alleviation, disease recurrence, response to treatment, response to other external or internal factors, conditions, or stressors, etc. Advantageously, the change in the prediction, diagnosis and/or prognosis of said disease in the subject may be monitored in the course of a medical treatment of said subject, preferably a medical treatment aimed at treating said disease. Such monitoring may be comprised, e.g., in decision making whether a patient may be discharged or needs further hospitalization or treatment. Typically, this is done by measuring the CD146 level in a subject at different time points during the stay in the ED, wherein a decrease in CD146 level in function of time indicates the condition of the subject is improving or has improved, white an increase of the CD146 level in function of time indicates the condition of the subject has worsened or is worsening.

The terms “sample” or “biological sample” as used herein include any biological specimen obtained from a subject. Samples may include, without limitation, whole blood, plasma, serum, red blood cells, white blood cells (e.g., peripheral blood mononuclear cells), saliva, urine, stool (i.e., faeces), tears, sweat, sebum, nipple aspirate, ductal lavage, tumour exudates, synovial fluid, cerebrospinal fluid, lymph, fine needle aspirate, amniotic fluid, any other bodily fluid, cell lysates, cellular secretion products, inflammation fluid, semen and vaginal secretions.

Preferred samples may include ones comprising CD146 in detectable quantities. In preferred embodiments, the sample may be whole blood or a fractional component thereof such as, e.g., plasma, serum, or a cell pellet. According to a specific embodiment, the sample used in the method according to the invention is blood or a fractional component thereof, advantageously plasma. Preferably the sample is readily obtainable by minimally invasive methods. Samples may also include tissue samples and biopsies, tissue homogenates and the like. Preferably, the sample used to detect CD146 levels is blood plasma. The term “plasma” defines the colorless watery fluid of the blood that contains no cells, but in which the blood cells (erythrocytes, leukocytes, thrombocytes, etc.) are suspended, containing nutrients, sugars, proteins, minerals, enzymes, etc.

According to a specific embodiment, the method of the invention further comprises measuring in the sample of the subject the presence or absence and/or quantity of one or more other biomarkers useful for diagnosing, predicting and/or prognosticating said disease, advantageously natriuretic peptides, more advantageously B-type natriuretic peptide (BNP), pro-B-type natriuretic peptide (proBNP), amino terminal pro-B-type natriuretic peptide (NTproBNP), mid-regional pro-atrial natriuretic peptide (MR-proANP) and/or troponin T.

As used herein, the terms “pro-B-type natriuretic peptide” (also abbreviated as “proBNP”) and “amino terminal pro-B-type natriuretic peptide” (also abbreviated as “NTproBNP”) and “B-type natriuretic peptide” (also abbreviated as “BNP”) refer to peptides commonly known under these designations in the art. As further explanation and without limitation, in vivo proBNP, NTproBNP and BNP derive from natriuretic peptide precursor B preproprotein (preproBNP). In particular, proBNP peptide corresponds to the portion of preproBNP after removal of the N-terminal secretion signal (leader) sequence from preproBNP. NTproBNP corresponds to the N-terminal portion and BNP corresponds to the C-terminal portion of the proBNP peptide subsequent to cleavage of the latter C-terminally adjacent to amino acid 76 of proBNP. As detailed above in relation to CD146, these terms encompass such peptides from any organism, including those with a native sequence, or variants and fragments thereof.

Exemplary human proBNP peptide includes without limitation the peptide from amino acid position 27 to position 134 of the natriuretic peptide precursor B preproprotein sequence as annotated under the NIH Entrez Protein (http://www.ncbi.nlm.nih.gov/sites/entrez?db=protein) accession number NP_002512 (version NP_002512.1 revised Jan. 25, 2009). The sequence of NP_002512 is shown in FIG. 3A (SEQ ID NO: 3) of WO2011/048168 and the exemplary sequence of proBNP from NP_002512 is shown in FIG. 3B (SEQ ID NO: 4) of WO2011/048168. Exemplary human NTproBNP peptide includes without limitation the peptide from amino acid position 27 to position 102 of the natriuretic peptide precursor B preproprotein sequence as annotated under said NIH Entrez Protein accession number NP_002512. The exemplary sequence of NTproBNP from NP_002512 is shown in FIG. 3C (SEQ ID NO: 5) of WO2011/048168. Exemplary human BNP peptide includes without limitation the peptide from amino acid position 103 to position 134 of the natriuretic peptide precursor B preproprotein sequence as annotated under said NIH Entrez Protein accession number NP 002512. The exemplary sequence of BNP from NP_002512 is shown in FIG. 3D (SEQ ID NO: 6) of WO2011/048168. See also Sudoh et al. 1989 (Biochem Biophys Res Commun 159: 1427-1434) for further exemplification of human preproBNP-derived peptides, including proBNP, NTproBNP and BNP. See also Maisel et al. 2008 (Eur J Heart Fail 10(9): 824-39) and Miller et al. 2007 (Biomarkers Med 1(4): 503-512) on using natriuretic peptide levels in clinical practice.

In the methods taught herein, the quantity of CD146 and/or the presence or absence and/or quantity of the one or more other biomarkers may be measured by any suitable technique such as may be known in the art.

According to specific embodiments of the methods and kits of the invention, the quantity of CD146 and/or the presence or absence and/or quantity of one or more other biomarkers is measured using a binding agent capable of specifically binding to CD146 and/or to fragments thereof, and a binding agent capable of specifically binding to said one or more other biomarkers, advantageously using an immunoassay technology, such as direct ELISA, indirect ELISA, sandwich ELISA, competitive ELISA, multiplex ELISA, radioimmunoassay (RIA) or ELISPOT technologies, or using a mass spectrometry analysis method or using a chromatography method, or using a combination of said methods.

In an embodiment, the quantity of CD146 and/or the presence or absence and/or quantity of the one or more other biomarkers may be measured using, respectively, a binding agent capable of specifically binding to CD146 and/or to fragments thereof, and a binding agent capable of specifically binding to said one or more other biomarkers.

In a preferred embodiment, said binding agent is capable of binding both the membrane-bound and plasma circulating forms of CD146. Preferably, said binding agent is capable of specifically binding or detecting the plasma circulating form of CD146.

In an embodiment, the binding agent may be an antibody, aptamer, photoaptamer, protein, peptide, peptidomimetic or a small molecule.

In a further embodiment, the quantity of CD146 and/or the presence or absence and/or quantity of the one or more other biomarkers is measured using an immunoassay technology, such as direct ELISA, indirect ELISA, sandwich ELISA, competitive ELISA, multiplex ELISA, radioimmunoassay (RIA) or ELISPOT technologies, or using a mass spectrometry analysis method or using a chromatography method, or using a combination of said methods.

According to another aspect, the present invention concerns a kit for diagnosing, predicting and/or prognosticating a disease associated with variations of the vascular wall tension comprising means for measuring the quantity of CD146, advantageously a binding agent thereof, more advantageously an antibody, and its use for said purpose.

According to a preferred embodiment, said kit comprises a reference control obtained from a subject not suffering of said disease or having a known diagnosis, prediction and/or prognosis of said disease.

According to another embodiment, said kit comprises means for detecting and/or measuring one or more other biomarkers useful for diagnosing, predicting and/or prognosticating said disease, advantageously B-type natriuretic peptide (BNP), pro-B-type natriuretic peptide (proBNP), amino terminal pro-B-type natriuretic peptide (NTproBNP), mid-regional pro-atrial natriuretic peptide (MR-proANP) and/or troponin T.

In an embodiment, any one of said one or more binding agents may be advantageously immobilised on a solid phase or support.

Said kit can also comprise a known quantity or concentration of CD146 and/or a fragment thereof, e.g. for use as controls, standards and/or calibrators. It can also comprise means for collecting the sample from the subject.

All components may be suitably labelled as known form the skilled person.

Also disclosed are reagents and tools useful for measuring CD146 and optionally the one or more other biomarkers concerned herein.

Binding agents, antibodies, labeled antibodies and required reagents or devices for detecting and/or quantifying the biomarkers disclosed in the present application are known from the skilled person and disclosed in detail in WO 2011/048168. Moreover, kits and/or systems are commercially available, e.g.:
Measurements of NT-proBNP concentration can be analyzed using a 1-step sandwich chemiluminescent immunoassay (e.g. with Cobas (Roche Diagnostics) or Dimension Vista Flex (Siemens));
BNP can be measured using the ARCHITECT i2000SR system (Abbott Laboratories) and the AxSYM BNP-Microparticle Enzyme Immunoassay (Abbott Laboratories);
MR-proANP can be measured using immunoluminometric assay (B.R.A.H.M.S. AG);
Troponin T can be analyzed with the high-sensitive assay of Roche Diagnostics;
Concentrations of sCD146 can be determined by ELISA, e.g. with CY-QUANT ELISA sCD14 (Biocytex).

### Examples

The invention is further described in detail by reference to the following experimental examples. These examples are provided for purposes of illustration only, and are not intended to be limiting unless otherwise specified.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### BRIEF DESCRIPTION OF THE FIGURES

FIG. 1: Levels of soluble CD146 and NT-proBNP before and after venous stress test.

FIG. 2: Prevalence of severe stenosis, atrial fibrillation and pulmonary hypertension and their combination in MS patients.

FIG. 3: Plasma biomarker levels according to severity of mitral stenosis, pulmonary hypertension and atrial fibrillation

A/ plasma levels of BNP [pg/ml], MR-proANP [pmol/l] and sCD146 [ng/ml] in patients with moderate or severe stenosis. B/ plasma levels of the 3 biomarkers in MS patients with or without pulmonary hypertension. C/ biomarker levels in presence or absence of atrial fibrillation.

FIG. 4: Correlation of biomarker values to systolic pulmonary artery pressure. The diagrams illustrate the correlation between systolic pulmonary pressure [sPAP, in mmHg] and the biomarkers BNP [pg/ml], MR-proANP [pmol/l], sCD146 [ng/ml]. Circles denote MS patients, crosses PAH patients.

FIG. 5: Plasma biomarker levels according to etiology of pulmonary hypertension. The figure illustrates plasma levels of BNP [pg/ml], MR-proANP [pmol/l] and sCD146 [ng/ml] in patients with pulmonary hypertension secondary to mitral stenosis (post-capillary) and pre-capillary pulmonary arterial hypertension.

FIG. 6: Plasma levels of BNP and sCD146 according to pulmonary congestion. Increasing radiological evidence of pulmonary congestion is associated with higher levels of sCD146. Plasma levels of BNP show higher dispersion and do not correlate with radiological pulmonary congestion.

FIG. 7: Plasma levels of BNP and sCD146 according to troponin levels. Increasing levels of troponin are associated with higher BNP but similar or lower levels of sCD146. Median and interquartile range are displayed.

### I/ EFFECT OF VASCULAR STRESS ON CD146

The venous stress mechanistic study aims at evaluating the role of peripheral congestion on plasma levels of sCD146 in chronic HF patients.

### Methods

### Subjects

A total of 44 chronic HF outpatients of Columbia University Medical Center (New York, USA) with left-ventricular ejection fraction (LVEF)<40%, NYHA functional class II or III without evidence of congestion on physical exam and on stable medical therapy underwent venous stress test according to a previously described protocol (Colombo P C et al., European Heart Journal. 2014; 35:448-454). Inclusion and exclusion criteria are summarized in Table 1:

TABLE 1
Inclusion and exclusion criteria for the “venous stress” study

| Inclusion criteria | Exclusion criteria |
|---|---|
| Age >18 years | Systolic blood pressure ≤90 mmHg |
| LVEF <40% | Unattainable peripheral venous |
| No evidence of peripheral venous | access in either forearm |
| congestion (no peripheral edema, | NYHA Class ≥ II angina |
| jugular venous distension <6 cm, | Heart transplant |
| negative hepatojugular reflex, no | Relevant renal dysfunction (Serum |
| ascites) | creatinine >2.5 mg/dl) |
| Patient receiving optimal medical | Acute infection |
| therapy, including ACEi or ARB | History of venous |
| and beta-blockers, as tolerated, | thromboembolism |
| for at least 4 weeks | Liver cirrhosis |
| | Pregnancy |

Peripheral venous stress without ischemia was created by inflating a pressure cuff around the dominant arm (=test arm), increasing venous arm pressure up to 30 mmHg above baseline. Blood was sampled through an indwelling venous catheter from the antecubital or basilic vein of the test arm after 90 minutes and from the control arm (lacking an inflated cuff) at baseline and after 90 minutes.

### Blood Samples Storage and Analysis

All samples were immediately centrifuged, and the aliquot portions of plasma and serum were stored in microcentrifuge tubes at −80° C. until assayed. Deidentified specimens were sent to independent core laboratories for analysis. Measurements of concentrations of NT-proBNP were performed at Lariboisiere University Hospital, Paris, France using a 1-step sandwich chemiluminescent immunoassay (Cobas, Roche Diagnostics, Basel, Switzerland). Concentrations of sCD146 were determined by ELISA (CY-QUANT ELISA sCD1460, Biocytex, France) at Lariboisiere University Hospital, Paris, France.

### Statistical Analysis

Continuous variables are expressed, after testing for normality using the Shapiro-Wilk test, as mean±standard deviation or median [interquartile range], as appropriate. Nominal variables are expressed as frequency (percentages). Wilcoxon signed rank test was used to examine the differences between test and control arm before and after venous congestion. Differences between two independent groups were assessed with the Wilcoxon rank sum test or Fisher's exact test, as appropriate. The null hypothesis was rejected with an adjusted two-sided p-value <0.05. Analyses were performed with the use of IBM SPSS Statistics, Version 21.0. (IBM Corp, Armonk N.Y., USA) and SAS, Version 9.2. (SAS Institute Inc, Cary N.C., USA).

### Ethical Considerations

The study was performed in observance of national laws and in accordance with the ethical standards of the Declaration of Helsinki, and was approved by local Ethical Committees. All patients provided written informed consent.

### Results

### Baseline Characteristics

Patients included in this study were clinically stable, predominantly middle-aged men with chronic HF with severely depressed LVEF. Cardiovascular risk factors were highly prevalent and ischemic heart disease accounted for at least a third of HF etiologies. Most patients were treated with disease-modifying therapies according to current guidelines (McMurray J J V et al., ESC Committee for Practice Guidelines. ESC Guidelines for the diagnosis and treatment of acute and chronic heart failure 2012: The Task Force for the Diagnosis and Treatment of Acute and Chronic Heart Failure 2012 of the European Society of Cardiology. Developed in collaboration with the Heart Failure Association (HFA) of the ESC. European Heart Journal. 2012; 33:1787-1847.)

Baseline characteristics of patients included in this study are summarized in Table 2:

### Impact of the Venous Stress on the Biomarker Levels

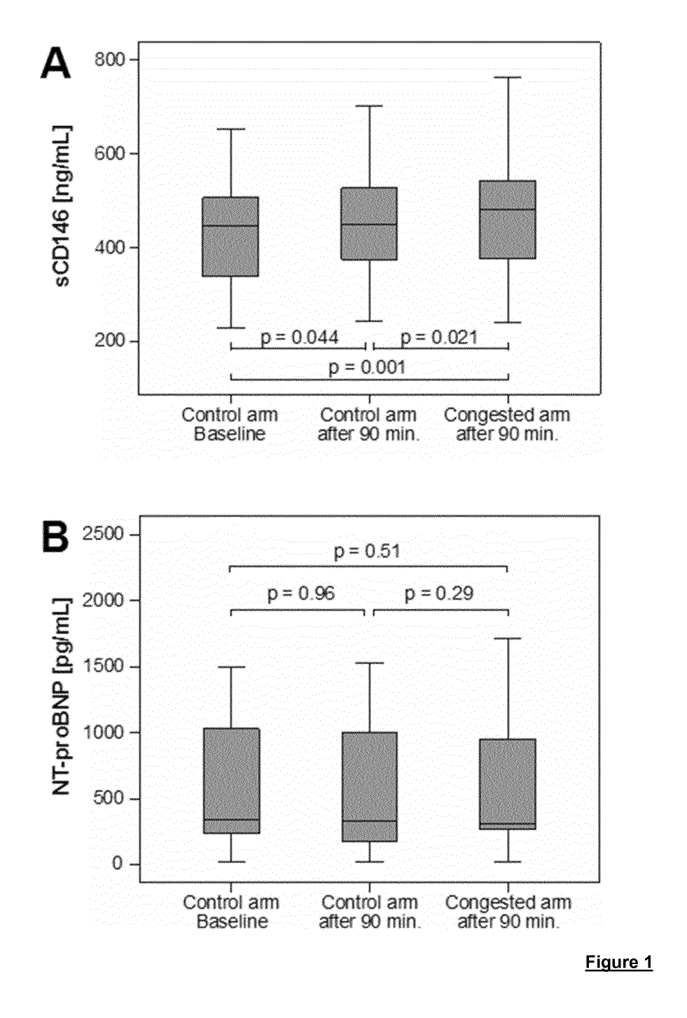

As shown in FIG. 1A, the induction of venous stress in stable HF patients was associated with an increase in circulating levels of sCD146 in the congested arm (481 [371-553] ng/ml) when compared to baseline (454 [339-507| ng/ml, p=0.001) and to the control arm (442 [374-527] ng/ml, p=0.02). Of note, sCD146 levels were also slightly higher in the control arm after 90 minutes compared to baseline (442 [374-527] vs. 454 [339-507| ng/ml, p=0.04).

By contrast, FIG. 1B illustrates plasma levels of NT-proBNP before and after congestion: no difference in levels of NT-proBNP in the congested arm (308 [218-1140] ng/ml) when compared to baseline (341 [191-1147| ng/ml, p=0.51) and to the control arm (327 [152-1137] ng/ml, p=0.29) was found.

### Conclusions

The present study confirmed high circulating sCD146, above normal values (Bardin N et al., Thromb Haemost. 2003; 90:915-920), in HF patients. An increase in pressure in forearm veins raised plasma levels of sCD146, but not of NT-proBNP, in the congested arm. A more modest but significant increase in plasma levels of sCD146 was also found in the contralateral arm (not exposed to congestion), likely due to a spillover from the congested arm into the bloodstream (Colombo P C et al., European Heart Journal. 2014; 35:448-454).

The present study demonstrates the increased release of sCD146 in presence of venous stress. To the knowledge of the inventors, this is the first demonstration of a biomarker specifically associated with peripheral venous congestion. Hence, in HF, endothelial sCD146 is a marker of congestion of the peripheral vasculature.

Cuff inflation during venous stress test not only promoted congestion, but necessarily reduced arterial perfusion pressure by impinging on the brachial artery. However, a reduction in perfusion pressure is a typical clinical feature of advanced HF where arterial blood pressure progressively declines (Barlera S, et al., Circ Heart Fail. 2013; 6:31-39), thus making this model even more relevant, from a pathophysiological standpoint. Of note, this human model of venous congestion has previously shown to mimic on a local scale, notable aspects of the phenotype that is typical of acute HF such as inflammation, neurohormonal and endothelial cell activation (Colombo P C et al., European Heart Journal. 2014; 35:448-454).

### II/ USE FOR THE ASSESSMENT OF MITRAL STENOSIS

Mitral stenosis (MS) may cause progressive dyspnea on exertion, pulmonary hypertension (PH), atrial fibrillation and right ventricular (RV) failure. Patients with MS presenting with change in dyspnea severity often require a complete cardiologic assessment, but the use of biomarkers may be an alternative for the initial assessment of MS and its complications. The aims of this study are to evaluate the role of several cardiovascular biomarkers for this purpose.

### Methods

### Study Population

Consecutive, clinically stable patients with moderate (valve area between 1.5 and 2 cm²) or severe (valve area <1.5 cm²) MS were included in the study between Jan. 1, 2011 and Mar. 31, 2015. This prospective multicenter observational study was performed at University Hospital of Besancon (France), Bichat University Hospital in Paris (France), and Cumhuriyet University Hospital of Sivas (Turkey). Exclusion criteria were: age <18 years; left ventricular ejection fraction <40%; recent acute coronary syndrome or documented coronary disease; combination of MS with severe aortic valve disease or more than mild mitral regurgitation; recent (<1 month) cardiac decompensation. Patients with high heart rate (>110 beats per minute), uncontrolled hypertension (systolic blood pressure >180 mmHg), severe anemia (hemoglobin <9 g/dL) or pregnancy were excluded as well.

One small cohort (n=16) of patients with pre-capillary PH (PAH) examined at University Hospital of Besancon was included for comparison of biomarker levels in different etiologies of pulmonary hypertension.

### Echocardiographic Study

All patients underwent transthoracic echocardiography at inclusion, and measurements were made in accordance with the recommendations of the European Association for Echocardiography (Baumgartner H, et al. (2009) EAE/ASE recommendations for clinical practice. Eur J Echocardiogr 10:1-25. doi: 10.1093/ejechocard/jen303). Severity of mitral valve stenosis (MS) was assessed by planimetric evaluation in the parasternal short axis view. MS was considered severe if the valve area was ≤1.5 cm² and moderate if the valve area was between 1.5 and 2 cm². Extent of mitral regurgitation was estimated by color Doppler and classified depending on the degree of incursion of the flow into the left atrium. Left ventricular (LV) systolic function was evaluated by measurement of the LV ejection fraction by the Simpson's biplane method. The left atrial size was assessed by measuring its area from the apical four chamber view or its volume in both the four and two chamber views. The left atrium (LA) was considered to be dilated if area was >20 cm², or volume >60 mL, or diameter >40 mm. Pulmonary pressures were calculated from maximal tricuspid regurgitation velocity on continuous Doppler in the apical four chamber view, taking into account the size and compliance of the inferior vena cava. A diagnosis of PH was retained for a tricuspid regurgitation velocity >2.8 m/sec, corresponding to systolic pulmonary artery pressure (PAP) >30 mmHg. Right ventricular (RV) function was assessed by measuring RV fractional area change (RV fac), tricuspid annular plane systolic excursion (TAPSE) and systolic myocardial velocity at the lateral tricuspid annulus (TAPSE S′). RV dysfunction was defined as the alteration of any one of these three criteria (i.e. RV fac <40%, TAPSE <18 mm; TAPSE S′<12 cm/sec).

### Biomarker Testing

Samples of venous blood were drawn on the same day as echocardiography was performed. Samples were centrifuged within 10 minutes in a refrigerated centrifuge, and stored at −80° C.

BNP was measured using the ARCHITECT i2000 system (Abbott Laboratories, Chicago, Ill., USA). MR-proANP was measured using immunoluminometric assay (B.R.A.H.M.S. AG, Hennigsdorf, Germany) and sCD146 using the CY-QUANT ELISA sCD146 kit (Biocytex, Asnieres, France).

### Statistical Analysis

Continuous variables are expressed as median (interquartile range), nominal variables as frequency (percentages). Group characteristics were compared with non-parametric tests: Fisher's exact test or the Mann-Whitney U-test for two groups, as appropriate; Chi-square or the Kruskall-Wallis H-test for three groups, as appropriate. For statistically significant differences between the groups, subsequent pairwise comparisons were performed using Dunn's procedure with Bonferroni correction of the p-value for multiple comparisons. Corrected p-values are reported. Correlation analysis was performed using Spearman's correlation coefficient. The diagnostic performance of all three biomarkers was assessed by receiver operating characteristic (ROC) analysis and expressed as area under the curve (AUC). The null hypothesis was rejected with an adjusted two-sided p-value <0.05. All analyses were performed with the use of IBM SPSS Statistics, Version 21.0. (IBM Corp, Armonk N.Y., USA).

### Ethical Considerations

The study was conducted in accordance with the principles of the Declaration of Helsinki and local national laws and was approved by the local Ethical Committee. All patients provided written informed consent. The study is registered with ClinicalTrials.gov under the number NCT01374880.

### Results

### Study Population

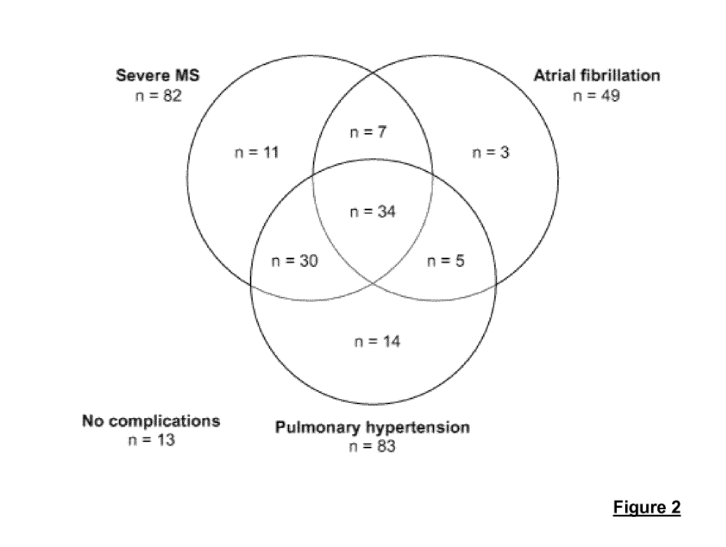

A total of 117, predominantly women (n=96, 72%) with mitral stenosis (MS) were included. In 82 patients (70%) the stenosis was classified as severe and moderate in the other 35 patients (30%). The median planimetric area was 1.34 cm² (IQR 1.1-1.58 cm²) and left atria were dilated in the majority of patients (n=104, 89%) with a median diameter of 49 mm (42-54 mm) and a volume of 101 ml (75-135 ml). Pulmonary hypertension (n=83, 71%) and atrial fibrillation (n=49, 42%) were common among MS patients (FIG. 2).

Table 3 summarizes the baseline characteristics of the MS patients:

TABLE 3
Baseline characteristics of the studied patients

| | Mitral stenosis | | PAH | | |
|---|---|---|---|---|---|
| | (n = 117) | | (n = 16) | | p-value |
| Demographics | | | | | |
| Age \|years] | 55 | (46-67) | 64 | (53-71) | 0.10 |
| Male gender | 21 | (18%) | 8 | (50%) | 0.008 |
| Weight [kg] | 73 | (60-82) | 61 | (49-89) | 0.31 |
| Height [cm] | 160 | (155-166) | 168 | (158-173) | 0.052 |
| BMI [kg/m2] | 27.8 | (23.5-31.3) | 23.6 | (19.1-28.2) | 0.01 |
| Hypertension | 37 | (32%) | 6 | (38%) | 0.78 |
| NYHA class | | | | | |
| I | 26 | (22%) | 3 | (19%) | |
| II | 58 | (50%) | 5 | (31%) | |
| III | 32 | (27%) | 2 | (12%) | |
| IV | 1 | (1%) | 6 | (38%) | |
| Treatment | | | | | |
| Betablockers | 61 | (52%) | 2 | (13%) | 0.003 |
| ACE-inhibitors or AT2 antagonist | 15 | (29%) | 4 | (25%) | 1.00 |
| Aldosterone antagonist | 34 | (29%) | 4 | (25%) | 1.00 |
| Diuretics | 57 | (49%) | 13 | (81%) | 0.017 |
| Digoxin | 14 | (28%) | 0 | (0%) | 0.016 |
| Vital signs | | | | | |
| Systolic blood pressure [mmHg] | 120 | (108-130) | 123 | (118-132) | 0.41 |
| Diastolic blood pressure [mmHg] | 71 | (65-81) | 70 | (69-78) | 0.76 |
| Heart rate [bpm] | 75 | (69-86) | 78 | (66-95) | 0.62 |
| Atrial fibrillation | 49 | (42%) | 2 | (13%) | 0.028 |
| Lab values | | | | | |
| Hemoglobin [g/dl] | 13.7 | (12.6-14.5) | 14 | (12.5-15) | 0.54 |
| eGFR [ml/min] | 75 | (59-90) | 65 | (57-97) | 0.52 |
| BNP [pg/ml] | 121 | (77-205) | 103 | (56-165) | 0.35 |
| MR-proANP [pmol/l] | 96 | (39-188) | 174 | (103-225) | 0.054 |
| sCD146 [ng/ml] | 320 | (263-440) | 562 | (322-620) | 0.003 |
| Echocardiography | | | | | |
| Left ventricle | | | | | |
| Septal thickness [mm] | 9 | (8-10) | 11 | (9-13) | 0.016 |
| PW-thickness [mm] | 9 | (8-10) | 10 | (9-11) | 0.032 |
| LV-EDD [mm] | 47 | (43-51) | 45 | (40-50) | 0.11 |
| LV-ESD [mm] | 32 | (29-35) | 26 | (19-33) | 0.002 |
| LV-EF [%] | 60 | (57-68) | 65 | (60-70) | 0.11 |
| Right ventricle | | | | | |
| RV dilation | 29 | (25%) | 14 | (87%) | <0.001 |
| RV dysfunction | 0 | (0%) | 5 | (31%) | <0.001 |
| Systolic PAP [mmHg] | 37 | (30-45) | 68 | (46-80) | <0.001 |
| Mitral valve | | | | | |
| Area (planimetry) [cm2] | 1.34 | (1.1-1.58) | 2.05 | (2-2.1) | <0.001 |
| Left atrium | | | | | |
| LA diameter [mm] | 49 | (42-54) | 39 | (32-43) | <0.001 |
| LA area \|cm2] | 33 | (28-38) | 18 | (16-20) | <0.001 |
| LA volume [ml] | 101 | (75-135) | . | (.—.) | — |

### Impact of Severity of MS, Pulmonary Hypertension and Atrial Fibrillation on Biomarkers

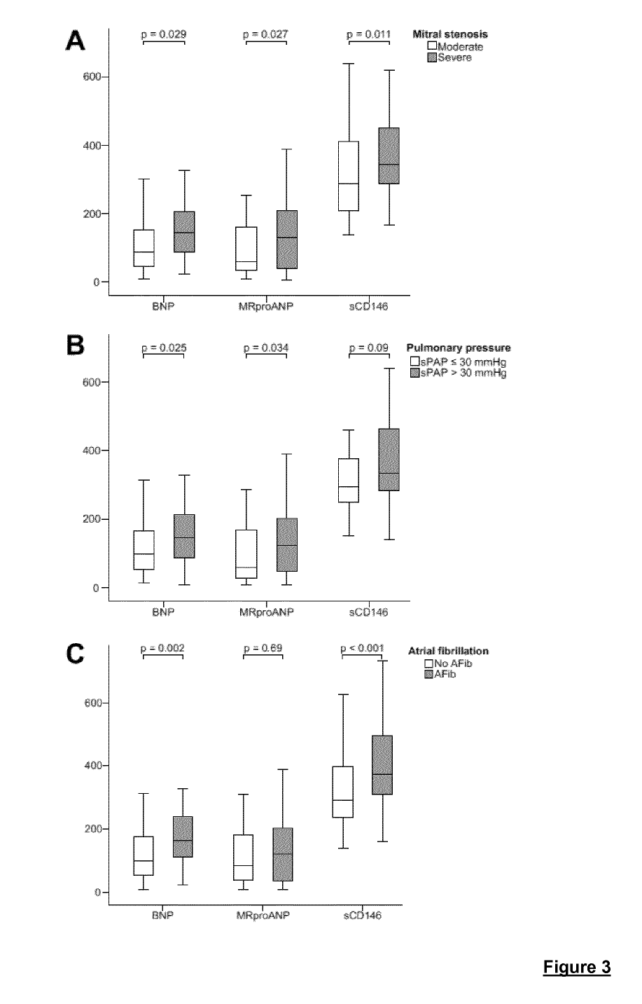

As shown in FIG. 3A, plasma levels of BNP (p=0.029), MR-proANP (p=0.027) and sCD146 (p=0.011) were higher in patients with severe MS compared to moderate MS.

As shown in Table 4 below, the area under the curve (AUC) after ROC analysis to discriminate between severe and moderate MS was between 0.63 and 0.65 for all three biomarkers:

TABLE 4
Receiver operating characteristics (ROC) curves of different
biomarkers for the assessment of complications of mitral stenosis

| | Area under the curve | |
|---|---|---|
| | (95% confidence interval) | |
| Biomarkers | Severe stenosis | Pulmonary hypertension |
| BNP [pg/ml] | 0.633 (0.512-0.754) | 0.636 (0.521-0.752) |
| MR-proANP [pmol/l] | 0.635 (0.525-0.744) | 0.629 (0.516-0.741) |
| sCD146 [ng/ml] | 0.653 (0.532-0.775) | 0.604 (0.490-0.717) |

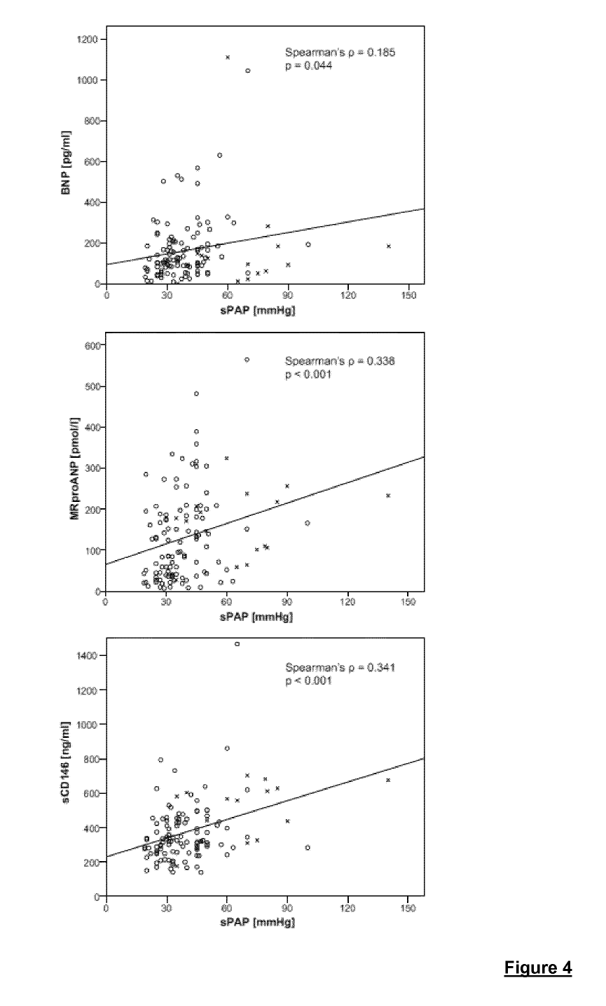

FIG. 3B illustrates that MS patients with systolic pulmonary pressure above 30 mmHg had higher levels of BNP (p=0.025) and MR-proANP (p=0.034) but no change of sCD146 levels compared to patients without PH. Systolic pulmonary artery pressure (sPAP) modestly correlated with BNP (p=0.185, p=0.044), MR-proANP (p=0.338, p<0.001), sCD146 (p=0.341, p<0.001), as shown in FIG. 4. The area under the curve (AUC) after ROC analysis to discriminate between sPAP≤30 mmHg and sPAP>30 mmHg was between 0.60 and 0.64 for all three biomarkers (Table 4).

As shown in FIG. 3C, plasma levels of BNP (p=0.002), and sCD146 (p<0.001) were higher in MS patients with atrial fibrillation compared to patients in sinus rhythm. However, the presence or not of atrial fibrillation did not influence plasma MR-proANP.

### Biomarkers in Pulmonary Hypertension

Among patients with elevated pulmonary artery pressures (n=99), the median systolic pulmonary artery pressure was 45 mmHg (35-50 mmHg) in MS patients with PH (n=83) and 68 mmHg (46-80 mmHg) in patients with pre-capillary PAH (p<0.001). Baseline characteristics of the cohort of patients with PAH (n=16) are summarized in Table 3.

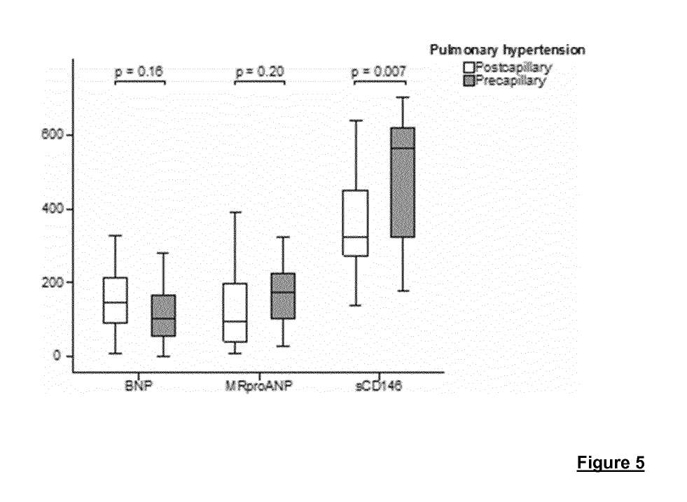

FIG. 5 illustrates plasma levels of all three biomarkers in MS patients with PH compared to levels in patients with PAH: PAH patients had higher levels of sCD146 compared to MS patients with PH (p=0.007), whereas no differences in BNP and MR-proANP were observed. Furthermore, among patients with elevated pulmonary artery pressures, RV dysfunction was present in 5 cases (5%) and associated with higher median levels of sCD146 (612 vs. 344 ng/ml, p=0.013) compared to patients with normal RV function, whereas no differences in BNP and MR-proANP levels were observed.

### Conclusions

Plasma levels of all three biomarkers were higher in severe MS compared to moderate MS. PH was associated with levels of BNP and MR-proANP. The presence of atrial fibrillation increased plasma levels of BNP and sCD146, whereas MR-proANP was not affected by atrial fibrillation. PAH patients had higher levels of sCD146 compared to MS patients with PH. RV dysfunction was associated with higher levels of sCD146.

In conclusion, mitral stenosis (MS) and its complications affect plasma levels of cardiovascular biomarkers. The use of MR-proANP may be useful for monitoring disease progression in MS, in particular for the assessment of severe stenosis and the presence of pulmonary hypertension in the early phase. sCD146 might help identifying patients with more advanced pulmonary hypertension and RV-dysfunction.

### III/ USE FOR THE ASSESSMENT OF PULMONARY CONGESTION

Aim of this study was the evaluation of sCD146 for the assessment of the degree of pulmonary congestion in the early phase of ACS.

### Methods

### Study Population

Consecutive patients with ACS admitted to the Coronary Care Unit (CCU) of the Cardiology Department of the University Hospital of Bmo (Czech Republic) from July 2009 to November 2012 were enrolled. The diagnosis of ACS was based on appropriate symptoms in conjunction with consistent changes on electrocardiogram, i.e. ST-segment elevation or depression, new left bundle branch block or negative T wave (Thygesen K, et al. Third universal definition of myocardial infarction. 2012. pp. 2551-67). Exclusion criteria were: age >85 years or estimated life expectancy due to non-cardiovascular reasons <12 months; known or newly diagnosed malignancy, inflammatory disease or connective-tissue disease; distance from the place of residence to the hospital of >100 km; absence of coronary stenosis with reduction of the intraluminal diameter >50% on coronary angiography.

### Blood Samples Storage and Analysis

Samples of venous blood for standard biochemical and hematological analyses as well as sCD146 were drawn immediately upon hospital admission before PCI. Troponin T and brain natriuretic peptide (BNP) were drawn exactly 24 hours after onset of chest pain. Samples were centrifuged within 10 min in a refrigerated centrifuge, and stored at −80° C. Troponin T was analyzed with the high-sensitive assay (Roche Diagnostics, Basel, Switzerland), BNP using the AxSYM BNP-Microparticle Enzyme Immunoassay (Abbott Laboratories, Chicago, Ill., USA), and sCD146 by ELISA (CY-QUANT ELISA sCD1460, Biocytex, France).

### Radiological Evaluation of Pulmonary Congestion

Pulmonary congestion was assessed by conventional chest radiography at admission.

Images were evaluated by certified radiologists and classified in 3 groups: no or mild congestion, interstitial pulmonary edema, alveolar pulmonary edema.

### Ethical Considerations

Written informed consent was obtained from all subjects before participation in the study. The study was performed in observation of national laws and in accordance with the ethical standards of the Declaration of Helsinki, and was approved by the Ethics Committee of Faculty Hospital Brno (Brno, Czech Republic).

### Statistical Analysis

Values are expressed as median (interquartile range, IQR) or as number (percentage), as appropriate. Three groups were compared with the Chi-square or the Kruskall-Wallis H-test, as appropriate. For statistically significant differences between the groups, subsequent pairwise comparisons were performed using Dunn's procedure with Bonferroni correction of the p-value for multiple comparisons. The null hypothesis was rejected with an adjusted two-sided p-value <0.05. All analyses were performed with the use of IBM SPSS Statistics, Version 21.0. (IBM Corp, Armonk N.Y., USA).

### Results

A total of 1021 patients presenting with ACS were prospectively screened. Patients without chest radiography at admission (n=94; 9%) were excluded from this analysis. Baseline characteristics of the 927 patients included in the study are summarized in Table 5:

TABLE 5
Baseline characteristics of the studied patients

| | | | No or mild | | Interstitial | | Alveolar | | |
|---|---|---|---|---|---|---|---|---|---|
| | Total | | congestion | | edema | | edema | | p- |
| | N = 927 | | N = 835 | | N = 72 | | N = 20 | | value |
| Age [years] | 61 | (55-67) | 61 | (55-67) | 63 | (55-68) | 70 | (59-73) | 0.010 |
| Male gender | 707 | (76%) | 643 | (77%) | 50 | (69%) | 14 | (70%) | 0.281 |
| Height [cm] | 174 | (168-179) | 174 | (168-180) | 170 | (168-179) | 170 | (165-174) | 0.120 |
| Weight [kg] | 85 | (75-95) | 85 | (75-95) | 8 | (76-90) | 83 | (74-97) | 0.582 |
| Systolic blood | 140 | (120-160) | 140 | (120-160) | 135 | (113-155) | 135 | (103-151) | 0.071 |
| pressure [mmHg] | | (120-160) | | | | | | | |
| Diastolic blood | 80 | (70-90) | 80 | (70-90) | 80 | (70-90) | 70 | (63-80) | 0.008 |
| pressure [mmHg] | | | | | | | | | |
| Heart rate [/min] | 76 | (66-87) | 75 | (66-86) | 82 | (74-105) | 87 | (73-98) | <0.001 |
| Type of acute | | | | | | | | | 0.022 |
| coronary syndrome | | | | | | | | | |
| Unstable angina | 36 | (4%) | 35 | (4%) | 1 | (1%) | 0 | (0%) | |
| NSTEMI | 278 | (30%) | 248 | (30%) | 18 | (25%) | 12 | (60%) | |
| STEMI | 613 | (66%) | 552 | (66%) | 53 | (74%) | 8 | (40%) | |
| Risk factors | | | | | | | | | |
| Hypertension | 508 | (55%) | 459 | (55%) | 35 | (49%) | 14 | (70%) | 0.224 |
| Dyslipidemia | 380 | (41%) | 339 | (41%) | 31 | (43%) | 10 | (50%) | 0.653 |
| Diabetes | 206 | (22%) | 171 | (21%) | 24 | (33%) | 11 | (55%) | <0.001 |
| Active smoking | 424 | (46%) | 384 | (46%) | 34 | (49%) | 6 | (30%) | 0.597 |
| Family history | 192 | (28%) | 179 | (29%) | 11 | (24%) | 2 | (20%) | 0.718 |
| Previous myo- | 107 | (12%) | 99 | (12%) | 7 | (10%) | 1 | (5%) | 0.562 |
| cardial infarction | | | | | | | | | |
| Previous PCI | 83 | (9%) | 80 | (10%) | 2 | (3%) | 1 | (5%) | 0.125 |
| Previous CABG | 22 | (2%) | 18 | (2%) | 4 | (6%) | 0 | (0%) | 0.149 |
| Previous stroke | 49 | (5%) | 38 | (5%) | 9 | (13%) | 2 | (10%) | 0.010 |
| Peripheral artery | 53 | (6%) | 41 | (5%) | 7 | (10%) | 5 | (25%) | <0.001 |
| disease | | | | | | | | | |
| COPD | 39 | (4%) | 35 | (4%) | 2 | (3%) | 2 | (10%) | 0.362 |
| Atrial fibrillation | 25 | (3%) | 19 | (2%) | 3 | (4%) | 3 | (15%) | 0.002 |
| Laboratory values | | | | | | | | | |
| at admission | | | | | | | | | |
| Hemoglobin | 143 | (133-153) | 143 | (133-153) | 142 | (129-157) | 137 | (113.5-149) | 0.170 |
| [g/L] | | | | | | | | | |
| Leucocytes | 10.9 | (8.7-13.7) | 10.7 | (8.6-13.3) | 12.4 | (9.7-16.5) | 13.4 | (10.9 17.8) | <0.001 |
| [G/L] | | | | | | | | | |
| Sodium | 140 | (137-141) | 140 | (137-141) | 139 | (137-141) | 138 | (137-142) | 0.196 |
| [mmol/L] | | | | | | | | | |
| Potassium | 4 | (3.7-4.4) | 4 | (3.7-4.4) | 4.1 | (3.8-4.4) | 4.5 | (4.1-5.0) | 0.002 |
| [mmol/L] | | | | | | | | | |
| Glucose | 7.6 | (6.3-10) | 7.5 | (6.2-9.7) | 9 | (7.8-12.1) | 13 | (7.5-17.1) | <0.001 |
| [mmol/L] | | | | | | | | | |
| Creatinine | 82 | (71-97) | 82 | (70-96) | 84 | (75-98) | 103 | (81-131) | 0.002 |
| [μmol/L] | | | | | | | | | |
| Troponin T* | 1.41 | (0.372-3.78) | 1.34 | (0.33-3.52) | 3.47 | (1.22-7.00) | 1.16 | (0.52-3.66) | <0.001 |
| [μg/L] | | | | | | | | | |
| CRP* | 18 | (6-62) | 15 | (6-51) | 70 | (24-175) | 66 | (39-163) | <0.001 |
| [mg/L] | | | | | | | | | |
| Length of stay | 5 | (4-7) | 5 | (4-7) | 6 | (4-9) | 7 | (5-12) | 0.007 |
| [days] | | | | | | | | | |
| In-hospital | 10 | (1.1%) | 4 | (0.5%) | 4 | (5.6%) | 2 | (10.0%) | <0.001 |
| mortality | | | | | | | | | |

Legend: BNP brain natriuretic peptide - CABG coronary artery bypass graft surgery - COPD chronic obstructive pulmonary disease - CRP C-reactive protein - NSTEMI non-ST-elevation myocardial infarction - PCI percutaneous coronary intervention - STEMI ST-elevation myocardial infarction.
*Reported BNP and Troponin T values are at 24 hours after admission, CRP values are at 48 h after admission

Patients were subsequently classified in 3 groups according to the degree of pulmonary congestion on chest radiography at admission: No or mild congestion (n=835), interstitial edema (n=72), alveolar edema (n=20). Ninety-two (10%) patients showed signs of pulmonary edema on chest radiography.

Patients with severe pulmonary congestion presented more often with NSTEMI, were older, had higher prevalence of diabetes, atrial fibrillation and peripheral artery disease, had higher creatinine levels and lower diastolic blood pressure at admission compared to the other subgroups. Patients with no or mild congestion had lower heart rate, lower inflammation parameters and lower glucose compared to patients with severe congestion. Of note, interstitial edema was associated with higher troponin levels compared to the other subgroups.

Patients with pulmonary congestion (interstitial or alveolar edema) had increased in-hospital mortality compared to patients without or with mild pulmonary congestion (5.6% and 10.0% vs. 0.5%, p<0.001). There was also a trend toward longer hospital stay with increasing pulmonary congestion, although pairwise comparisons were not statistically significant.

For the overall population, median level of sCD146 was 320 ng/mL (IQR 251-398 ng/mL, range 95-2866 ng/mL) and the median level of BNP was 263 pg/mL (IQR 125-473 pg/mL, range 10-11567 pg/mL).

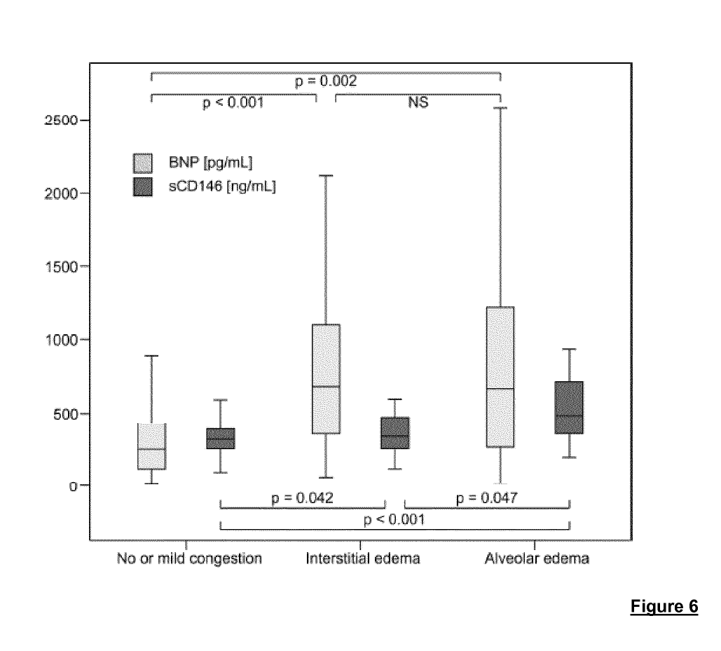

FIG. 6 shows that median plasma levels of BNP were higher in patients with interstitial (679 pg/mL, IQR 355-1097 pg/mL) or alveolar (665 pg/mL, IQR 267-1214 pg/mL) pulmonary edema compared to patients without or with mild signs of congestion (251 pg/mL, IQR 119-430 pg/mL). No difference between patients with interstitial and alveolar pulmonary was found (p-values shown in FIG. 6).

FIG. 6 further shows that plasma levels of sCD146 were better associated with radiological evidence of pulmonary congestion than BNP with stepwise increase in circulating sCD146 with increasing degree of pulmonary congestion: median plasma levels of sCD146 in patients without or with mild signs of congestion, interstitial edema and alveolar edema were 316 ng/mL (IQR 249-388 ng/mL), 348 ng/mL (IQR 267-478 ng/mL) and 438 ng/mL (IQR 346-690 ng/mL), respectively. Moreover, plasma levels of sCD146 showed a lower variability compared to BNP.

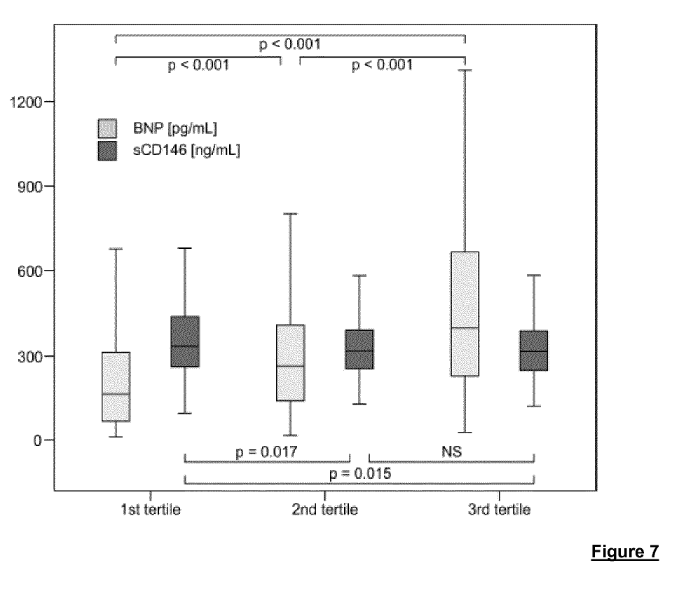

FIG. 7 shows the relationship of BNP, sCD146 and myocardial necrosis. BNP levels were associated with the level of troponin. Splitting the population in 3 groups according to troponin levels, BNP values were higher in the second and third tertiles of troponin compared to the first tertile (p<0.001). In contrast to BNP, sCD146 levels were not increased, and even slightly decreased, in the second and third tertiles of troponin compared to baseline.

### Conclusions

Plasma levels of sCD146 well reflected radiological evidence of pulmonary congestion: the higher plasma levels of sCD146, the worse the degree of pulmonary congestion. Interestingly, in contrast to BNP, sCD146 levels were not affected by the level of troponin. In other words, the novel endothelial biomarker sCD146 correlates with radiological evidence of pulmonary congestion in the early phase of ACS and, in contrast to BNP, is not affected by the amount of myocardial cell necrosis.

Since relevant pulmonary congestion complicates the course of one of ten patients presenting with ACS and, as confirmed in our study, negatively influences short-term outcome, sCD146 may help emergency physicians, cardiologists and intensivists to assess and monitor pulmonary congestion in patients with ongoing ACS.

1. (canceled)

2. A method for treating a disease associated with variations of the vascular wall tension in a subject, wherein the method comprises:
measuring the quantity of CD 146 in a sample from the subject; and
treating the subject with a prophylactic or therapeutic treatment selected from the group consisting of drug treatment and surgical treatment.

3. The method of claim 2, wherein said disease is a mitral valve disease.

4. The method of claim 2, wherein said disease is organ congestion, wherein the subject does not have clinical sign of heart disease.

5-6. (canceled)

7. The method according to claim 2, wherein the sample is blood or a fractional component thereof.

8. The method according to claim 2, wherein the method further comprises measuring in the sample of the subject the presence or absence and/or quantity of one or more other biomarkers selected from the group consisting of B-type natriuretic peptide (BNP), pro-B-type natriuretic peptide (proBNP), amino terminal pro-B-type natriuretic peptide (NTproBNP), mid-regional pro-atrial natriuretic peptide (MR-proANP) and troponin T.

9. The method according to claim 2, wherein the quantity of CD 146 is measured using a binding agent capable of specifically binding to CD 146 and/or to fragments thereof.

10-14. (canceled)

15. The method of claim 9, wherein the measuring comprises an immunoassay technology, a mass spectrometry analysis method, a chromatography method, or a combination of the foregoing.

16. The method of claim 3, wherein the mitral valve disease-is mitral stenosis or mitral regurgitation.

17. The method of claim 2, further comprising:
re-measuring the quantity of CD146 in a sample from the subject after the treatment; and
discharging the patient if the quantity of CD146 has decreased.

18. The method of claim 2, further comprising:
re-measuring the quantity of CD146 in a sample from the subject after the treatment; and
providing further treatment if the quantity of CD146 has increased.

19. The method of claim 2, wherein the treatment comprises administering a drug selected from the group consisting of a diuretic drug, an anticoagulant, a beta blocker, an ACE inhibitor and a calcium channel blocker.

20. The method of claim 2, wherein the treatment comprises valve repair.

21. The method of claim 4, wherein the organ congestion is pulmonary or visceral congestion.
